(19) United States
(12) Patent Application Publication
(10) Pub. No.: US 2023/0306620 A1
(43) Pub. Date: Sep. 28, 2023

(54) Live Angiography Registration

(71) Applicant: LightLab Imaging, Inc., Westford, MA (US)

(72) Inventors: Angela Zhang, Stow, MA (US); Shimin Li, Acton, MA (US); Robert Steinbrecher, Wrentham, MA (US); Jack Fince, Manchester, NH (US); Paul Gervasio, North Billerica, MA (US)

(73) Assignee: LightLab Imaging, Inc., Westford, MA (US)

(21) Appl. No.: 18/125,520

(22) Filed: Mar. 23, 2023

Related U.S. Application Data
(60) Provisional application No. 63/477,877, filed on Dec. 30, 2022.
(60) Provisional application No. 63/323,335, filed on Mar. 24, 2022.

(51) Int. Cl. G06T 7/33; G06T 3/40; G06T 3/20

(52) U.S. Cl. CPC G06T 7/337; G06T 3/40; G06T 3/20; G06T 2207/30101; G06T 2207/20092; G06T 2207/10121; G06T 2207/10081; G06T 2207/10088; G06T 2207/10101; G06T 2207/10132; G06T 2207/10108; G06T 2207/20212

(57) ABSTRACT

The disclosure relates to methods of providing live x-ray images such as fluoroscopic images in combination with reference information co-registered angiography frames that are motion corrected in real time. In part, the disclosure describes an improvement on the existing workflow where the user must remember where the vessel features from OCT are and use a reference frame to estimate the location of desired stent placement. Disclosed herein are methods to correlate the live imaging feed and the reference angiography image such that the user may dynamically zoom and pan the live imaging feed and the reference angiography image.

### CROSS REFERENCE TO RELATED APPLICATIONS

The present application claims the benefit of the filing date of U.S. Provisional Patent Application No. 63/323,335, filed on Mar. 24, 2022, and U.S. Provisional Patent Application No. 63/477,877, filed on Dec. 30, 2022, the disclosures of which are hereby incorporated herein by reference.

### BACKGROUND

The disclosure relates generally to the field of intravascular imaging and data collection systems and methods.

Coronary artery disease is one of the leading causes of death worldwide. The ability to better diagnose, monitor, and treat coronary artery diseases can be of life saving importance. During some diagnostic procedures, various medical and technical personnel are viewing multiple sources of information including angiography images and fluoroscopy images as part of stent planning or stent delivery. Switching between these two sources of data can be fatiguing and often requires personnel to hold images and other data associations in their mind as they switch from a changing fluoroscopic view to an angiographic view as part of stent planning and delivery. Further, manipulating the live angiography feed and the reference image can be cumbersome and lead to inaccurate associations between the two images.

### BRIEF SUMMARY

The present disclosure describes a method to register angiography images from different time points, with and without contrast, so that features from the contrast image can be mapped accurately onto the non-contrast image. This will enable the user to directly view contrast and pullback features in the non-contrast image, reducing the error from using a reference frame only.

The disclosure relates to methods comprising using one or more processors to access a live imaging feed of a blood vessel, receiving a reference image of the blood vessel, aligning the live imaging feed and the reference images such that objects represented in a field of view of the reference image correspond to objects represented in a field of view of the live imaging feed, receiving user input associated with the reference image, adjusting the field of view of the reference image based on the user input, and automatically adjusting the field of view of the live imaging feed based on the user input to the reference image. According to aspects of the disclosure, the user input may be a selected zoom level.

According to aspects of the disclosure, the method may further comprise aligning the live imaging feed and the reference image using one or more processors identifying an area of interest on the reference image, calculating a scale factor based on a size of the objects represented in the field of view of the reference image and a size of the objects represented in the field of the live imaging feed, wherein the fields of view are associated with a grid, identifying a point of origin of the grid associated with the live imaging feed, transposing the point of origin into the reference image, calculating a distance coordinate based on the distance between the area of interest and the transposed point of origin, and converting the coordinates of the area of interest to match the coordinates of the live imaging feed, based on the scale factor and distance coordinate. The scale factor may be based on the selected zoom level and is calculated as a ratio of a zoomed reference image and a non-zoomed reference image. According to aspects of the disclosure, the method may further comprise using one or more processors to simultaneously displaying the converted area of interest on the reference image based on the converted coordinates and live imaging feed.

According to aspects of the disclosure, the object may include at least a portion of a blood vessel. The live imaging feed may comprise an extravascular image derived from an imaging system based on angiography, fluoroscopy, x-ray, nuclear magnetic resonance, or computer aided tomography. The reference image may comprise a still extraluminal image co-registered with intravascular data, wherein the intravascular data may be derived from an intravascular device that collects data using at least one of optical coherence tomography (OCT), intravascular ultrasound, near-infrared spectroscopy, or micro-OCT.

According to aspects of the disclosure, the reference frame may further comprise a surgical guide overlay. In adjusting the reference image and the live imaging feed, the user input may comprise simultaneously panning the live imaging feed and the reference image.

The disclosure relates to a system comprising one or more processors configured to access a live imaging feed of a blood vessel, receive a reference image of the blood vessel, align the live imaging feed and reference image such that objects represented in a field of view of the reference image correspond to objects represented in a field of view of the live imaging feed, receive user input associated with the reference image, adjust the field of view of the reference image based on the user input, and simultaneously adjust the field of view of the live imaging feed based on the user input to the reference image. The user input may comprise a selected zoom level.

According to aspects of the disclosure, the one or more processors may be further configured to identify a selected area of interest on the reference image, calculate a scale factor based on a size of the objects represented in the field of view of the reference image and a size of the objects represented in the field of view of the live imaging feed, wherein the fields of view are associated with a grid, identify a point of origin of the grid associated with the live imaging feed, transposing the point of origin onto the reference image, calculate a distance coordinate based on the distance between the area of interest and the transposed point of origin and convert coordinates of selected area of interest to match coordinates of the live imaging feed, based on the scale factor and the distance coordinate. The scale factor may be based on the selected zoom level and is calculated as a ratio of a zoomed reference image and a non-zoomed reference image. According to aspects of the disclosure, the one or more processors may be further configured to simultaneously display the converted area of interest on the reference image based on the converted coordinates and the live imaging feed.

The disclosure relates to methods of providing live x-ray images such as fluoroscopic images in combination with reference information co-registered angiography frames that are motion corrected in real time. In part, the disclosure describes an improvement on the existing workflow where the user must remember where the vessel features from OCT are and use a reference frame to estimate the location of desired stent placement. Disclosed herein is a method where motion corrected OCT information will be displayed directly on top of the fluoroscopy feed to the user.

One aspect of the disclosure relates to a method of providing annotations on a live angiography feed, comprising identifying, through an intravascular imaging procedure, one or more portions of a patient's vessel, matching a frame of the live angiography feed with a frame from the intravascular imaging procedure, such that both frames are captured during corresponding portions of a cardiac cycle of a patient, correcting rigid displacement, using rigid registration, and identifying, based on the matched frames and the corrected breathing motion, a spot on image overlay that corresponds to a portion of the patient's vessel shown in the live angiography feed and the one or more portions identified during the intravascular imaging technique.

The method may further comprise the frame from the intravascular imaging procedure is a co-registered frame of intraluminal data and extraluminal data.

The method may further comprise the intraluminal data is derived from an OCT probe or an IVUS probe.

The method may further comprise the extraluminal data is derived from an imaging system based on angiography, fluoroscopy, x-ray, nuclear magnetic resonance, or computer aided tomography.

The method may further comprise determining the cardiac cycle of the patient using cardiovascular timing parameters, pressure signals and heartbeat.

The method may further comprise the rigid registration includes accounting for rigid displacement of the intravascular data, wherein the rigid displacement is caused by breathing of the patient, movement of the patient, movement of the machinery, or shifting of the camera.

The method may further comprise matching the frame in the live angiography feed with the frame from the intravascular imaging procedure are predicted by artificial technology techniques.

The method may further comprise placing markers on the live angiography feed. The method may further comprise correlating the coordinates of the live angiography feed and the frame from the intravascular imaging procedure to facilitate a simultaneous zoom feature.

Another aspect of the disclosure relates to a data collection system comprising a probe configured to capture intraluminal data, an imaging device configured to capture extraluminal image data, a computing device configured to receive the intraluminal data and extraluminal image data, wherein the computing device is configured to identify, through an intravascular imaging procedure, one or more portions of a patient's vessel, match a frame in the live angiography feed with a frame from the intravascular imaging procedure, such that both frames are captured during corresponding portions of a cardiac cycle of the patient, correct rigid displacement, using rigid registration, and identify, based on the matched frames and the corrected breathing motion, a spot on an image overlay that corresponds to a portion of the patient's vessel shown in the live angiography feed and the one or more portions identified during the intravascular imaging technique.

The system may further comprise the intraluminal data is derived from an OCT probe or an IVUS probe.

The system may further comprise the extraluminal data is derived from an imaging system based on angiography, fluoroscopy, x-ray, nuclear magnetic resonance, or computer aided tomography.

The system may further comprise the computing device is configured to further determine the cardiac cycle of the patient using cardiovascular timing parameters, pressure signals, and heartbeat.

The system may further comprise the rigid registration comprises accounting for rigid displacement of the intravascular data.

The system may further comprise the rigid displacement is caused by breathing of the patient, movement of the patient, movement of the machinery, or shifting of the camera.

The system may further comprise matching the frame in the live angiography feed with the frame from the intravascular imaging procedure are predicted by artificial technology techniques.

The system may further comprise the computing device is configured to further place markers on the live angiography feed. The system may further comprise the computing device is configured to correlate the coordinates of the live angiography feed and the frame from the intravascular imaging procedure to facilitate a simultaneous zoom feature.

Another aspect of the disclosure relates to a non-transitory computer-readable medium storing instructions executable by one or more processors for processors for performing a method of providing annotation on a live angiography feed, comprising, identifying, through an intravascular imaging procedure, one or more portions of a patient's vessel, matching a frame in the live angiography feed with a frame from the intravascular imaging procedure, such that both frames are captured during corresponding portions of a cardiac cycle of the patient, correcting rigid displacement, using rigid registration, and identifying, based on the matched frames and the corrected breathing motion, a spot on an image overlay that corresponds to a portion of the patient's vessel shown in the live angiography feed and the one or more portions identified during the intravascular imaging technique.

Another aspect of this disclosure relates to a methods for simultaneously zooming on a live angiography feed and a reference image, comprising identifying, using one or more processors, a first point of origin on the live angiography feed, wherein the point of origin is the center of the live angiography feed, identifying, using one or more processors, a second point of origin on the reference image, wherein the second point of origin is different from the first point of origin, calculating, using one or more processors, a scale factor based on a selected zoom level, converting, using one or more processors, a selected coordinate of the reference image using the scale factor, determining, using one or more processors, the distance between the converted selected coordinate and the center of the reference image, and aligning, using one or more processors, the live angiography feed coordinates and the reference image coordinates based on the distance to the center from the converted selected coordinate.

### BRIEF DESCRIPTION OF THE DRAWINGS

FIG. 1 an example system according to aspects of the disclosure.

FIG. 2 is a flow diagram illustrating a method of correction in image analysis according to aspects of the disclosure.

FIG. 3 shows are example graphs illustrating tip location data according to aspects of the disclosure.

FIG. 4 are example graphs illustrating beats per minute (BPM) prediction data according to aspects of the disclosure.

FIGS. 5A-5B illustrate examples of automatic angiography segmentation, according to aspects of the disclosure.

FIG. 6 shows an example of a visualization of the phase matched frames between live angiography and contrast data.

FIG. 7 shows an example of rigid displacement according to aspects of the disclosure.

FIG. 8 shows an example of rigid motion correction according to aspects of the disclosure.

FIG. 9 is a flow chart illustrating an example method of stent mapping and deployment according to aspects of the disclosure.

FIG. 10 shows fluoroscopy and reference angiography frames with OCT pullback features side by side.

FIG. 11 is an example of the deployment screen according to aspects of the disclosure.

FIG. 12 is an example of the deployment screen according to aspects of the disclosure.

### DETAILED DESCRIPTION

The present disclosure provides for displaying annotations marking vessel features and/or a location for desired stent placement on a live angiography feed. The annotations may be identified during an intravascular imaging procedure, such as a pullback of an intravascular imaging probe. Angiography images may be registered from different time points, with and without contrast, so that features from the contrast image can be mapped accurately onto the non-contrast image. A user may directly view contrast and pullback features in the non-contrast image, reducing the error from using a reference frame only. The present disclosure also provides for methods to correlate the live angiography feed and the reference angiography image such that the user may dynamically zoom and pan the live angiography feed and the reference angiography image.

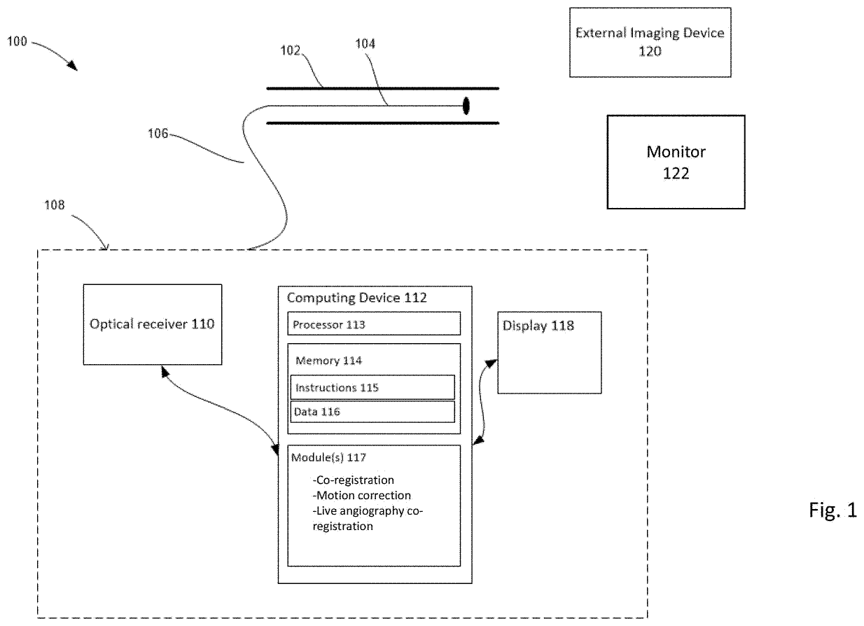

FIG. 1 illustrates a data collection system 100 for use in collecting intravascular and extravascular data. The system may include a data collection probe 104. The data collection probe 104 may be, for example, an OCT probe, an IVUS catheter, micro-OCT probe, near infrared spectroscopy (NIRS) sensor, or any other device that can be used to image a blood vessel 102. In some examples, the data collection probe 104 may be a pressure wire, a flow meter, etc. While the examples provided herein refer to an intravascular imaging device, such as an OCT probe, the use of an OCT probe is not intended to be limiting. For example, an IVUS catheter, a pressure wire, or another intravascular data collection device may be used in conjunction with or instead of the OCT probe. A guidewire, not shown, may be used to introduce the probe 104 into the blood vessel 102. The probe 104 may be introduced and pulled back along a length of a blood vessel while collecting data. The intravascular data sets, or frames of image data, may be used to identify features, such as vessel narrowing due to plaque, calcium deposits, etc.

OCT is a catheter-based imaging modality that uses light to peer into coronary artery walls and generate images thereof for study. Utilizing coherent light, interferometry, and micro-optics, OCT can provide video-rate in-vivo tomography within a diseased vessel with micrometer level resolution. Viewing subsurface structures with high resolution using fiber-optic probes makes OCT especially useful for minimally invasive imaging of internal tissues and organs. This level of detail made possible with OCT allows a user to diagnose as well as monitor the progression of coronary artery disease. IVUS imaging uses high-frequency sound waves to create intravascular images.

Intravascular imaging of portions of a patient's body provides a useful diagnostic tool for doctors and others. For example, intravascular imaging of coronary arteries may reveal the location of a narrowing or stenosis. This information helps cardiologists to choose between an invasive coronary bypass surgery and a less invasive catheter-based procedure such as angioplasty or stent delivery.

The probe 104 may be connected to a subsystem 108 via an optical fiber 106. The subsystem 108 may include a light source, such as a laser, an interferometer having a sample arm and a reference arm, various optical paths, a clock generator, photodiodes, and other OCT, IVUS, micro-OCT, NIRS, and/or pressure wire components.

The probe 104 may be connected to an optical receiver 110. According to some examples, the optical receiver 110 may be a balanced photodiode-based system. The optical receiver 110 may be configured to receive light collected by the probe 104. The probe 104 may be coupled to the optical receiver 110 via a wired or wireless connection.

The system 100 may further include, or be configured to receive data from, an external imaging device 120. The external imaging device may be, for example, an imaging system based on angiography, fluoroscopy, x-ray, nuclear magnetic resonance, computer aided tomography, etc. The external imaging device 120 may be configured to noninvasively image the blood vessel 102. According to some examples, the external imaging device 120 may obtain one or more images before, during, and/or after a pullback of the data collection probe 104.

The external imaging device 120 may be in communication with subsystem 108. According to some examples, the external imaging device 120 may be wirelessly coupled to subsystem 108 via a communications interface, such as Wi-Fi or Bluetooth. In some examples, the external imaging device 120 may be in communication with subsystem 108 via a wire, such as an optical fiber. In yet another example, external imaging device 120 may be indirectly communicatively coupled to subsystem 108 or computing device 112. For example, the external imaging device 120 may be coupled to a separate computing device (not shown) that is in communication with computing device 112. As another example, image data from the external imaging device 120 may be transferred to the computing device 112 using a computer-readable storage medium.

The subsystem 108 may include a computing device 112. One or more steps may be performed automatically or without user input to navigate images, input information, select and/or interact with an input, etc. In some examples, one or more steps may be performed based on receiving a user input by mouse clicks, a keyboard, touch screen, verbal commands, etc.

The computing device may include one or more processors 113, memory 114, instructions 115, data 116, and one or more modules 117.

The one or more processors 113 may be any conventional processors, such as commercially available microprocessors. Alternatively, the one or more processors may be a dedicated device such as an application specific integrated circuit (ASIC) or other hardware-based processor. Although FIG. 1 functionally illustrates the processor, memory, and other elements of device 110 as being within the same block, it will be understood by those of ordinary skill in the art that the processor, computing device, or memory may actually include multiple processors, computing devices, or memories that may or may not be stored within the same physical housing. Similarly, the memory may be a hard drive or other storage media located in a housing different from that of device 112. Accordingly, references to a processor or computing device will be understood to include references to a collection of processors or computing devices or memories that may or may not operate in parallel.

Memory 114 may store information that is accessible by the processors, including instructions 115 that may be executed by the processors 113, and data 116. The memory 114 may be a type of memory operative to store information accessible by the processors 113, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions 115 and data 116 are stored on different types of media.

Memory 114 may be retrieved, stored, or modified by processors 113 in accordance with the instructions 115. For instance, although the present disclosure is not limited by a particular data structure, the data 116 may be stored in computer registers, in a relational database as a table having a plurality of different fields and records, XML documents, or flat files. The data 116 may also be formatted in a computer-readable format such as, but not limited to, binary values, ASCII or Unicode. By further way of example only, the data 116 may be stored as bitmaps comprised of pixels that are stored in compressed or uncompressed, or various image formats (e.g., JPEG), vector-based formats (e.g., SVG) or computer instructions for drawing graphics. Moreover, the data 116 may comprise information sufficient to identify the relevant information, such as numbers, descriptive text, proprietary codes, pointers, references to data stored in other memories (including other network locations) or information that is used by a function to calculate the relevant data.

The instructions 115 can be any set of instructions to be executed directly, such as machine code, or indirectly, such as scripts, by the processor 113. In that regard, the terms “instructions,” “application,” “steps,” and “programs” can be used interchangeably herein. The instructions can be stored in object code format for direct processing by the processor, or in any other computing device language including scripts or collections of independent source code modules that are interpreted on demand or compiled in advance. Functions, methods and routines of the instructions are explained in more detail below.

The modules 117 may include a co-registration module and a live angiography co-registration module. According to some examples, the modules may include software such as preprocessing software, transforms, matrices, and other software-based components that are used to process image data or respond to patient triggers to facilitate co-registration of different types of image data by other software-based components or to otherwise perform such co-registration. The modules can include lumen detection using a scan line based or image based approach, stent detection using a scan line based or image based approach, indicator generation, stent expansion evaluation and assessment, stent landing zone detection and indication for deployed stents; angiography and intravascular imaging co-registration, and other modules supportive and programmed to perform the methods disclosed herein.

The computing device 112 may be adapted to co-register intravascular data with a luminal image. For example, computing device 112 may access co-registration module to co-register the intravascular data with the luminal image. The luminal image may be an extraluminal image, such as an angiograph, x-ray, or the like. The co-registration module may co-register intravascular data, such as an intravascular image, pressure readings, virtual flow reserve (“VFR”), fractional flow reserve (“FFR”), resting full-cycle ration (“RFR”), flow rates, etc. with the extraluminal image. In some examples, the co-registration module may co-register intravascular data with an intraluminal image, such as an intraluminal image captured by an OCT probe, IVUS probe, micro-OCT probe, or the link.

In one example, the co-registration module may co-register intraluminal data captured during a pullback with one or more extraluminal images. For example, the extraluminal image frames may be pre-processed. Various matrices such as convolution matrices, Hessians, and others can be applied on a per pixel basis to change the intensity, remove, or otherwise modify a given angiography image frame. As discussed herein, the preprocessing stage may enhance, modify, and/or remove features of the extraluminal images to increase the accuracy, processing speed, success rate, and other properties of subsequent processing stages. A vessel centerline may be determined and/or calculated. In some examples, the vessel centerline may be superimposed or otherwise displayed relative to the pre-processed extraluminal image. According to some examples, the vessel centerline may represent a trajectory of the collection probe 104 through the blood vessel during a pullback. In some examples, the centerline may be referred to as a trace. Additionally or alternatively, marker bands or radiopaque markers may be detected in the extraluminal image frames. According to some examples, the extraluminal image frames and the data received by collection probe 104 may be co-registered based on the determined location of the marker bands.

The system may include a monitor 122. The monitor 122 may include, without limitation, an electrocardiogram monitor configured to generate data relating to cardiac function and showing various states of the subject such as systole and diastole. Knowing the cardiac phase can be used to assist the tracking of vessel centerlines, as the geometry of the heart, including the coronary arteries, is approximately the same at a certain cardiac phase, even over different cardiac cycles. In part, the disclosure relates to systems and methods that use cardiovascular system timing parameters, and or signals such as electrocardiogram (ECG) and pressure signals, such as aortic pressure signals, to identify the angiography frames that correspond to parts of the heart cycle. The dicrotic notch and other timing indicia such as those used to identify systole and diastole can be used. Further, the disclosed systems and methods may identify angiography frames corresponding to a current fluoroscopy image based on real time correlation with such signals and/or timing parameters.

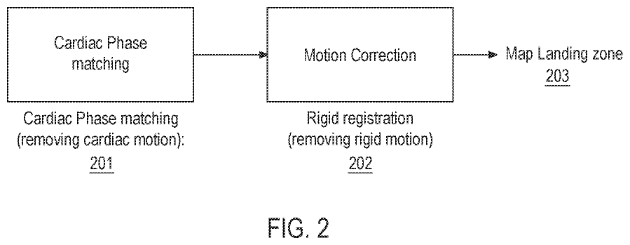

FIG. 2 is a flow diagram showing motion correction to be applied to the co-registered image. Particularly, a motion correction module may take into account cardiac motion, breathing motion, body movements, etc. prior to overlaying the co-registered image on a live angiography feed. The motion correction may be applied to the contrast image features to accurately overlay them on top of the live angiography feed.

According to some examples, for each live fluoroscopy frame, the corresponding angiography frame is identified based on signal or timing parameter correlation, such as through ECG correlation. Further, in some embodiments, intravascular probe markers, such as radio opaque markers, are used under angiography and correlated therewith as part of an intravascular imaging session, as described above. In this way, the intravascular imaging facilitates angiography correlation.

In a first phase, cardiac phase matching 201 is performed. During the cardiac phase matching 201, cardiac motion is removed. Cardiac motion relates to motion displayed on the images from the heart beating. In this phase, cardiac motion is removed from angiography co-registered image. In a second phase, rigid registration 202 is performed. During the rigid registration 202, rigid motion is removed. Rigid motion relates to breathing motion, such as the expansion and contraction of lungs patient movement during imaging. Additionally, any shifting of the camera over time may be approximated as rigid motion. In a third phase, after correcting angiography co-registered image for both cardiac activity and breathing/shifting motion for each frame, the stent landing zone can be mapped as well as intravascular imaging pullback features from the contrast angiography images onto the live angiography video feed. Each of these phases will be described, in turn below.

Due to the 2D projection of the 3D blood vessels in angiography, cardiac motion is deformable. In order to correct for cardiac motion, contrast images are identified that are in the same phase of the heartbeat cycle as an angiography frame being displayed at a particular instant in time during the display of the live angiography feed.

Correcting for cardiac motion may include predicting the beats per minute (BPM) of the patient. The BPM will allow for finding angiography frames from the intravascular imaging pullback which are phase-matched to the current non-contrast fluoroscopy frame.

There are at least two modalities with which to estimate BPM. In some examples, the modalities may be imaging modalities, such as OCT, angiography, IVUS, NIRS, μOCT, etc. For both modalities, a BPM prediction algorithm is based on custom autocorrelation values within a defined range of window sizes.

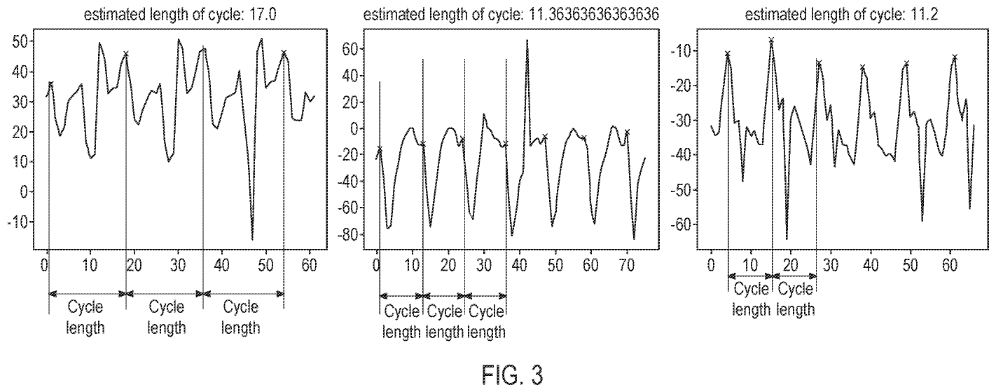

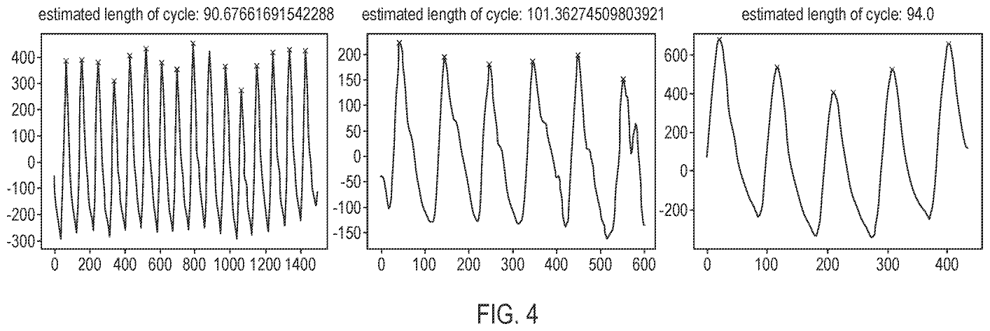

FIGS. 3 and 4 illustrate various examples of determining the cycle length, or BPM, or a patient. In some examples, a second modality may be aortic (AO) pressure data. AO pressure may have a defined cycle over the course of a heartbeat. The AO pressure may, in some examples, be used to determine, or predict, the BPM of a patient. As shown in FIGS. 3 and 4, the BPM may be determined based on points that are at the same or substantially the same location each cycle. The AO pressure data or other timing or signal data such as ECG may be used to determine what part of heart cycle corresponds to a given AO pressure data. According to some examples, the AO pressure data may correspond to identifying systole and diastole in heart cycle or other trackable time periods relative to a heart cycle.

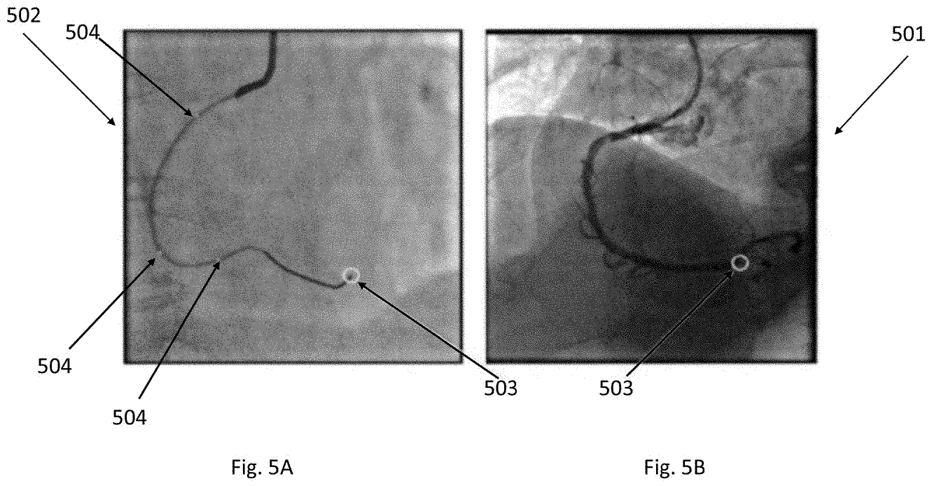

According to some examples, a first modality may be angiography video. According to some examples, vascular features and/or features of the imaging system may be visible in both contrast 501 (FIG. 5B) and non-contrast 502 (FIG. 5A) angiography images. The features of the imaging system may be, for example, the wire tip 503 and/or markers on the wire 504. The computing device may be configured to detect the features or segment an image of an artery or other structure into various component tissue types or regions of interest, through artificial intelligence (AI) systems, such as one or more machine learning models. In some examples, a machine learning model may be used to detect the features in the non-contrast images. The machine learning models may be trained using raw angiography images. In certain examples, the machine learning model may be trained using labelled image training data, for example angiography images with contrast and without contrast.

The AI system receives a plurality of training images, each training image annotated with locations of one or more features of the imaging system. The AI system can receive training image data labelled with locations of features of the imaging system detected in each image. As part of receiving the plurality of training images, the system can split the data into multiple sets, such as image frames for training, testing, and validation.

The AI system processes the plurality of training images to annotate each image. The AI system trains the machine learning model using the processed plurality of training images. In some examples, the model is configured to generate as output, a segmentation map representing predicted one or more regions of interest, including portions of the input image predicted to be features of the imaging system.

The machine learning model can be trained according to any technique for supervised learning, and in general training techniques using datasets in which at least some of the training examples are labelled. For example, the machine learning model can be one or more neural networks with model parameter values that are updates as a part of a training process using backpropagation with gradient descent, either on individual image frames or on batches of image frames, as examples.

In some examples, the machine learning model at least partially includes an encoder-decoder architecture with skip connections. As another example, the machine learning model can include one or more neural network layers as part of an autoencoder trained to learn compact (encoded) representations of images from unlabeled training data, such as images taken using OCT, IVUS, NIRS, OCT-NIRS, μOCT, or taken using any other of a variety of other imaging technologies. The neural network layers can be further trained on input training images as described herein, and the machine learning model can benefit from a broader set of training by being at least partially trained using unlabeled training images.

The training can be done using a loss function that quantifies the difference between a location predicted by the system for a feature of the imaging system, versus the ground-truth location for the feature of the imaging system received as part of the training data for the input image. The loss function can be, for example a distance between the predicted location and ground-truth location for a feature of the imaging system, measured at one or more pairs of points on the predicted and ground-truth location. In general, any loss function that compares each pixel between a training image with a predicted annotation and a training image with a ground-truth annotation can be used.

The system can perform training until determining that one or more stopping criteria have been met. For example, the stopping criteria can be a preset number of epochs, a minimum improvement of the system between epochs as measured using the loss function, the passing of a predetermined amount of wall-clock time, and/or until a computational budget is exhausted, e.g., a predetermined number of processing cycles.

Existing models can be augmented with existing machine learning models trained to predict non-lipid regions of interest, to also predict the locations of features of an imaging system, as one of a plurality of different output channels. The machine learning model can be trained to output multiple channels each corresponding to a respective region of interest, which can be expressed for example as one or more output images and/or as a segmentation map or other data characterizing regions of an input image as corresponding to different regions of interest.

In part, the AI system is designed such that it can be installed or combined with an imaging system such as an intravascular imaging system, an ultrasound system, or an x-ray system such as an angiography or fluoroscopy system. For example, the location of a given features, such as wire tip 503, may be plotted over time. By plotting the x and y locations of these features over time, the BPM of the patient over time can be estimated.

FIGS. 5A and 5B depict examples of automatic angiography segmentations. FIG. 5A illustrates features detected by the deep learning model in non-contrast images 502, as described above. The detected features may be, for example, wire tip 503 and marker locations 504. FIG. 5B illustrates features detected by the deep learning model in contrast images 501. The features may be, for example, wire tip 503.

According to some examples, an automatic angiography segmentation may be applied. For example, features detected by the machine learning model may be displayed in the non-contrast image 502. Such features may be displayed, for example, using a mask. As one example, different portions of the vessel may be identified using a mask with different colors, where each color corresponds to a segmented portion of the vessel. It should be understood that other types of masks may be applied, indicating any of a number of various features.

Once the AO pressure data or other timing data or signals is mapped to heart cycle or other clock or timing subsystem, then data of interest may be co-registered with pre-computed angiography data that has been co-registered with OCT/IVUS markers 504. In general, the systems and methods identify the heart cycle that tracks or corresponds with a particular frame of image data or other parameter of interest.

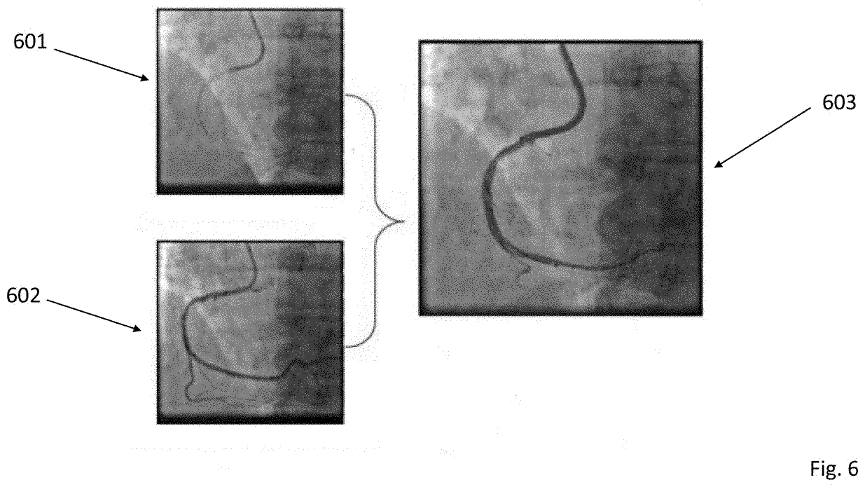

FIG. 6 illustrates an example of a phase matched image frame 603. The phased matched image frame 603 may include a matched live angiography image frame 601 and contrast image frame 602. Live angiography image frame 601 may correspond, or substantially correspond, to contrast angiography image frame 602. According to some examples, contrast angiography image frame 602 may include intravascular data obtained during the co-registration of the angiography image frame and an intravascular image frame. The heart cycle may contain repeating patterns, such as repeating pressure rises, dicrotic notches, and ECG pulses. These features of the live heart cycle data may be correlated, or matched, with the first set of heart cycle data.

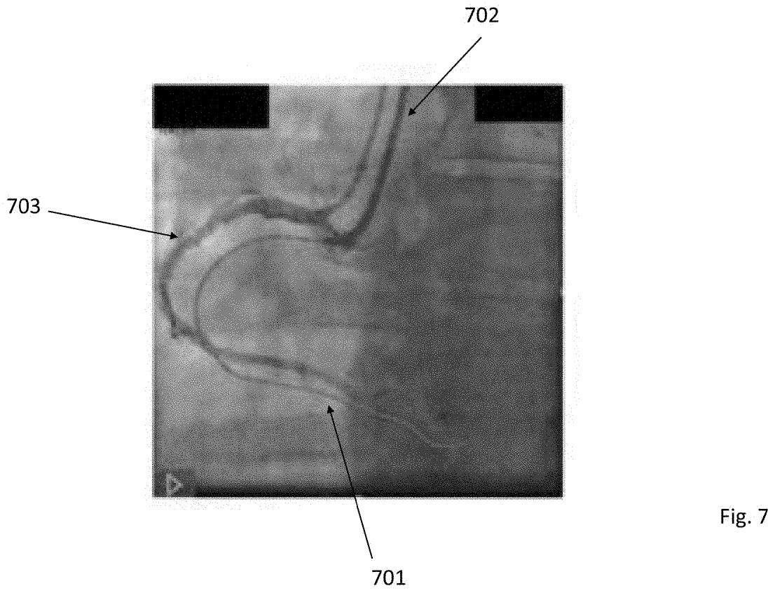

FIG. 7 illustrates rigid displacement where in the angiography image the location, or placement, of guide wire 701 path and the catheter 702 on the image does not align with the location of the blood vessel 703. Rigid displacement may be due, for example, to rigid movement. Specifically, rigid displacement may be caused by rigid motion such as breathing motion, such as the expansion and contraction of lungs patient movement during imaging, the camera shifting over time. To account for rigid displacement, a rigid transformation technique may be used to compensate, or correct, this type of motion. Features visible in both contrast and non-contrast angiography images are used for the rigid registration 202 step of the motion correction.

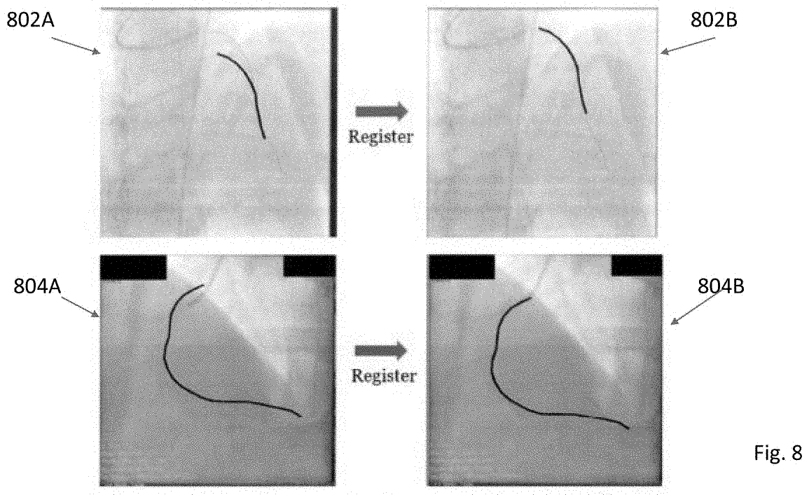

FIG. 8 illustrates angiography images after a rigid transformation has been applied. For example, images 802A and 804A illustrate non-contrast angiography images with a centerline overlaid. The centerline may be determined, for example, using an angiography image taken with contrast. As shown, the center line in each image 802A, 804A is offset from the vessel. The system may apply the rigid transformation technique to adjust the positioning of the vessel centerline based on the determined rigid displacement. As shown in images 802B, 804B, after the system applies the rigid transformation technique, the centerline may align, or substantially align, with the blood vessel in the image.

In one embodiment, the co-registered angiography and intravascular image may be combined, or co-registered, with the live angiography images or video such that intravascular markers, intravascular image data, intravascular data, and/or other parameters may be overlaid onto live angiography images or video. Stent detection, stent expansion, side branches, and other image data detected based on collected intravascular datasets can be linked to or displayed relative to the live angiography images and angiography images as a result of the co-registration between and among the three image datasets.

These various sets of data, extraluminal imaging, intravascular imaging, and live angiography may be combined, interlaced, used, juxtaposed or integrated in various ways to present combinations of co-registered data to end user. Angiography images may be registered from different time points, with and without contrast, so that features from the contrast image can be mapped accurately onto the non-contrast image. In this regard, diagnostic imaging can be performed using less contrast solution without sacrificing accuracy or image quality.

Using angiography images, fluoroscopy data, intravascular data, and/or other imaging modalities in support of cardiovascular diagnosis and stenosis treatment is of great value if it can be done on an expedited timescale and in a manner that helps the physician. Dealing with various competing obstacles to these goals represents important technical challenges. The use of co-registration data and signals, such as AO, ECG, systole transitions, diastole transitions, and others along with the generation of various interlaced, static, combined, and fused datasets, including streams of data that has a live subset of frames and a stored, historic, or otherwise non-live subset of frames can be used in various embodiments.

In part, the disclosure relates to systems and methods of overlaying angiography co-registration images and/or data onto live angiography. Further, the disclosure relates to systems and methods to combine angiography co-registration information with one or more live angiography feeds. Directly viewing the contrast and pullback features in the non-contrast live angiography feed will reduce the error from using the reference frame only.

Given that various data collection and diagnostic systems, such as angiography systems that use OCT, IVUS, or other intravascular imaging systems use imaging probes with radiopaque markers to track a given probe, angiography data can be co-registered with intravascular data. Both of these data sets can be co-registered, correlated, or cross-correlated with live angiography feeds using pressure signals used for monitoring aortic pressure or other pressure signals, EKG signals, dicrotic notch signals and location, and other timing signals.

An accurate map landing zone may be identified upon completion of the first and second phase. The map may be, for example, an overlay for the live angiography feed. Accordingly, an accurate landing zone may be one in which a marker will correspond to a correct location on the patient's vessel as depicted in the live angiography feed. While the cardiac phase matching and motion correction are described above as occurring in different phases, in other examples the cardiac phase matching and motion correction may be performed in a different order, at overlapping times, or simultaneously.

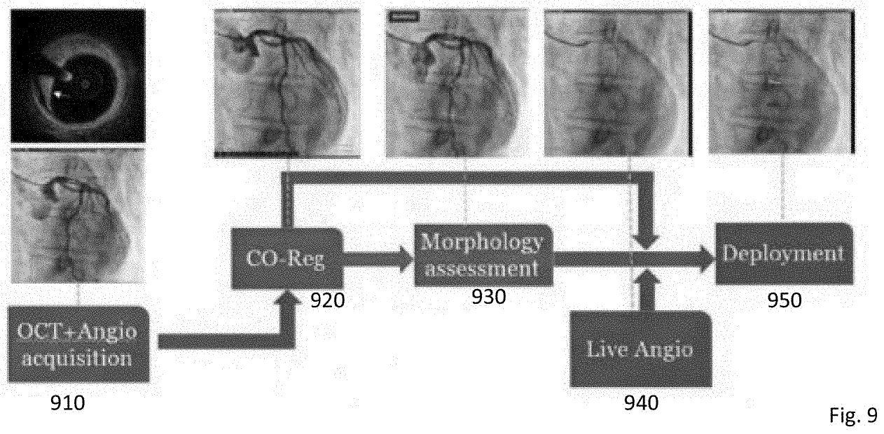

After correcting angiography co-registered image for both cardiac activity and breathing/shifting motion for each frame, the stent landing zone can be mapped as well as intravascular imaging pullback features from the contrast angiography images onto the live angiography video feed, shown in FIG. 9.

FIG. 9 illustrates an example method co-registering intravascular image(s) and/or image data with extraluminal images and then co-registering, or pairing, that co-registered image with a live angiography feed.

In block 910, intravascular image data and extravascular image data are obtained simultaneously. The intravascular image data may be obtained during a pullback. The intravascular image data may be OCT, IVUS, NIRS, etc. Extraluminal image data may be obtained before, during, and/or after the pullback. The extravascular image data may be angiography images. Both datasets are generated, transmitted, processed, and stored in a central computer. The images may be co-registered based on markers or a common point of reference.

In block 920, the intravascular image data may be co-registered with the extraluminal image data. After the data acquisition, a determination is made as to which angiography frame represents the closest reference point to the initial OCT frame. This determination may also be made either by a human user or an automatic computer routine. Either a human user or a computer algorithm may inspect the angiography data and estimate which frame corresponds to the initial frame of the pullback. For example, the computing device 112 may include one or more modules configured to automatically run and capture angiography images and tag each image by its acquisition time and support co-registration of intravascular image data tagged with an acquisition time. Further, the computing device 112 may include one or more modules configured to automatically detect a radio-opaque marker on each angiography image corresponding to the intravascular images. The co-registration module computes the intravascular imaging catheter's path on all angiography images corresponding to the intravascular image acquisition during the pullback of the probe through the vessel being imaged.

In one embodiment, side branches detected in OCT frames of data using OCT image processing can be used as an input to improve co-registration with angiography data. For example, in one embodiment, each OCT frame includes a flag (yes/no) indicating if a side branch is present. Further, once the co-registration is obtained, positions of stents, calcium deposits, lipid deposits, thrombus, thin capped fibroatheromas (TCFAs or “vulnerable plaques”), vessel normalization, side branch detection, FFR values (which may be computed as a vascular resistance ratio (VRR) values based on OCT image data), lumen size values, stents, and various other data described herein can be overlaid on the angiography image or the OCT images in light of the co-registration between the datasets.

In block 930, the system may perform a morphology assessment. In some examples, the morphology assessment may be cardiac motion and/or rigid displacement. To account for any identified cardiac motion, the system may phase match the image frames. In some examples, to account for any identified rigid displacement, the system may apply a rigid transformation technique.

In block 940, the system may pair the co-registered and corrected images with live angiography images and/or video. The corrected images may be, for example, co-registered intraluminal image data and extraluminal image data that have been phased matched and/or had the rigid displacement technique applied. In some examples, the co-registered and corrected images may be paired with a fluoroscopy video. Using the intravascular and extravascular image datasets stored on the memory device, the computing device may pair the co-registered images with the live fluoroscopy video using the same or similar reference points marked during co-registration. For example, the co-registered images and the live fluoroscopy video are paired using reference points such as distance from a point of interest, time captured, and/or markers.

In block 950, the system may output corrected image overlaid on the live angiography image and/or fluoroscopy video. According to some example, the output may include annotations shown in relation to the vessel displayed in the live angiography feed. The annotations may align, or substantially align, with locations identified during a previous intravascular imaging procedure for stent placement or other treatment.

The foregoing is useful when co-registering live angiography or fluoroscopy data. Specifically, a real time correlation is used that effectively identifies the part of the heart cycle that can be tracked over time relative to AO pressure data or other data or signals such ECG data for angiography data as well as corrects for rigid motion. As a result, this facilitates selecting any frame from set of angiography image frames and replacing such an angiography frame with a live angiography frame. A stent mapping or replacement can be used or a picture in picture representation can be used as well as others. Various interlacing techniques can be used. This frame swapping supports using a library of images from angiography that were previously generated with contrast.

In one embodiment, during an intravascular pullback image frames are dark under angiography. There is a discrete period of time for pullback, which can be tracked and indexed relative to angiography frames and registered relative to AO pressure data, ECG data, dicrotic notch, or other timing data. In one embodiment, every subsequent interlacing of frames can be performed using AO data or other timing data. One or more displays can be used to show live angiography data using a frame grabber to pull frames from live feed and interlace with angiography frames or combine with intravascular data. Intravascular imaging markers such as OCT/IVUS markers and other information can be combined with, overlaid upon or otherwise used with live angiography feed. This fusing or combining of various types of image data with live angiography data can be used to support various outcomes such as accurately knowing where the stent landing zone is positioned.

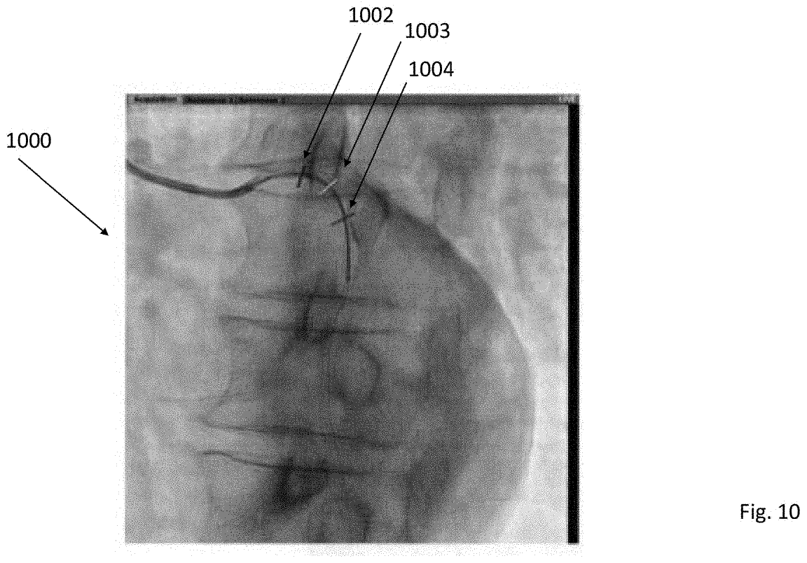

FIG. 10 illustrates an example of overlaying annotations from an intravascular imaging procedure onto the live angiography feed. Data from the co-registered image may be overlaid onto live angiographic images in accordance with aspects of the disclosure described above.

In this particular example, for example, FIG. 10, shows a live angiography image 1001 that is displayed with stent planning markers 1002, 1003, and 1004 having been overlaid onto the angiography image. The stent planning markers 1002, 1003, 1004 may correspond to annotations made by a physician during an intravascular imaging procedure. For example, the intravascular imaging procedure may be performed using an OCT, IVUS, or other type of probe. The procedure may identify portions of the patient's vessel having narrowings due to plaque, calcium deposits, etc.

These stent planning markers 1002-1004 may be based on markers from the OCT pullback mapped onto angiography images. The relative location of these markers will move from one angiographic image to another, as the patient's heart cycle will cause various arteries within the heart to move. However, by correlating both the intravascular image and angiographic image to the patient's heart cycle and rigid movement, the stent planning markers 1002-1004 can be overlaid onto each angiographic image in a manner that maintains their proper position relative to the artery for which the stent is to be placed. Thus, the system provides a more accurate placement of stent planning markers from intravascular imaging techniques onto a live angiography feed. These markers and other indicia help reduce geographic misplacement in procedures such as stent delivery and placement by ensuring physicians target the region they had planned to address in the intravascular imaging procedure.

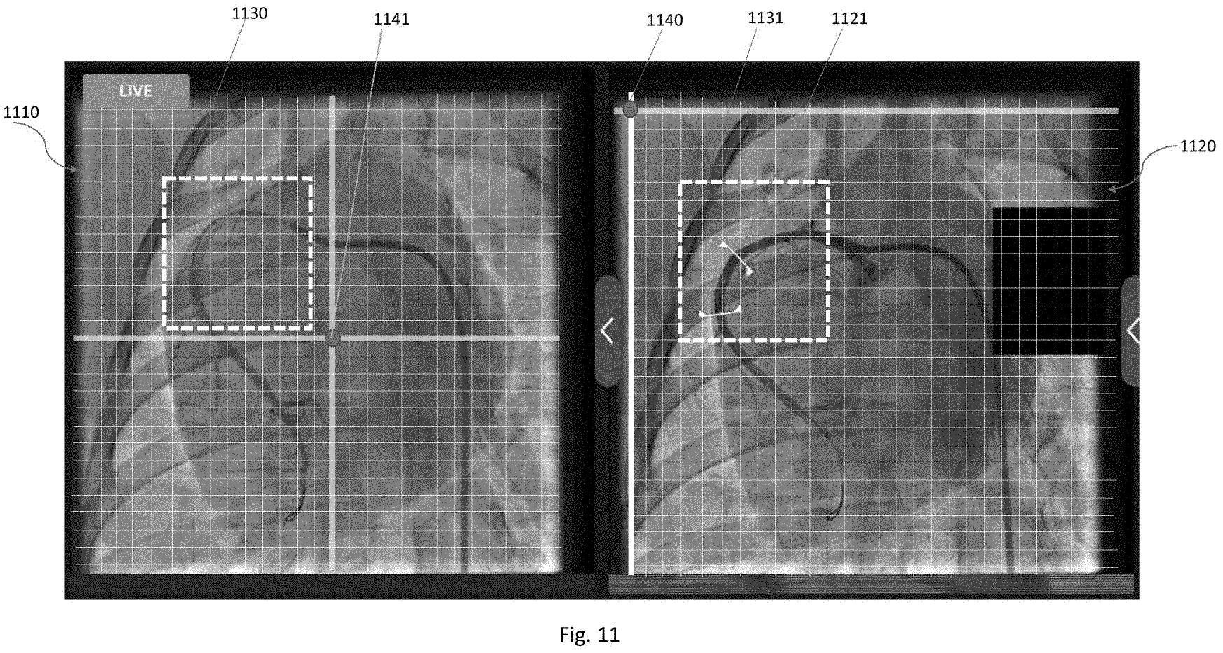

FIG. 11 illustrates a sample deployment screen with grid coordinates overlaid onto the screen. The left screen shows the live angiography feed 1110 of the X-ray fluoroscopy. The right screen shows the reference angiography image 1120 with the stent plan marked between the brackets 1121. Both images 1110 and 1120 reflect the field of view of imaging devices. The objects of the fields of view should correspond to each other. For example, he objects displayed in the live angiography feed should correspond to the objects displayed in the reference image. In some examples, the objects represented are portions of a blood vessel,

The system may place a grid layer over the displayed images. In some examples, the gird may be internal or visible to the user. The grid layers may be associated with the fields of view of each image, such that the grid layer remains constant as the images change based on the imaging device location. The grid layer may create coordinates for the images. Each grid layer may comprise a point of origin, from which the system will calculate the coordinates.

The squared off section on the display highlights the area of interest (AOI), 1130-1131, selected by the user. The reference angiography image 1120 with the stent plan 1121 overlay may be used as a guide to help with precise positioning of the device. The reference angiography image 1120 may be co-registered as described above. In some examples, the grid overlay would not be visible to the user but merely internal for the system to use.

The system may be equipped with a feature that may allow the user to zoom and pan the reference angiography image. This zoom feature may be capable of simultaneously zooming and panning the reference angiography image 1120 and the live angiography feed 1110. This will provide the user the ability to zoom in on the AOI in the live angiography feed 1110 while deploying an intravascular device, such as an interventional device. The system may be equipped with a processor with code to run the steps necessary to facilitate the zoom feature. In some examples, the grid layer may adjust based on the selection of the zoom feature. For example, if the user selected to magnify the image by 2, the grid layer may also be magnified to reflect the size of the image.

The system may be configured to correlate two images by converting the coordinates of a first image to match the coordinates of a second image. For example, to correlate the live angiography feed 1110 with the reference angiography image 1120, the system may convert the coordinates of the reference angiography image 1120 to match the coordinates of the live angiography feed 1110. Converting the coordinates may include determining points of origin as the base coordinate, or 0,0, of each image. In some examples, the images may have varying points of origin, such that the system must account for differing points of origin when correlating the two images. In the example shown in FIG. 11, the first point of origin 1141, or 0,0, of the live angiography feed 1110 is located at the center of the image. The point of origin 1140, or 0,0, of the reference angiography image 1120 is in the upper left corner of the image. It should be understood that in other examples, the points of origin for each image may be in different positions than shown in FIG. 11.

In some examples, the system is configured to receive a zoom input from the user. The zoom input may be how much the user wants to enlarge or lessen the images, such as the magnification number. For example, the user may select an AOI and decide to zoom in on the image and video screen 3x. The system may use the zoom input to calculate a scale factor. A scale factor may be calculated using a ratio between the new image and the original image. For example, for a 3× zoom input, the ratio would be 3:1 and the scale factor would be 3.

The user may select an area within either image to closely examine, such as the AOI 1131 of the reference angiography image 1120. The display may enlarge the area around the selected point, such as an enlarged image of the AOI. In some examples, the system may select two points within the user selected area, such as the upper left and lower right coordinates of the AOI. The selected points may be converted using the determined scale factor.

The selected points may be further converted from their absolute coordinates to distance coordinates. Where the system is correlating a first image to a second image, the absolute coordinates of the first image may be calculated relative to the point of origin of a first image, such as the second point of origin 1140 of the reference angiography image 1120. The distance coordinates of the first image may be calculated by determining the distance from each absolute coordinate to the point of origin of the second image, such that the distance coordinates of the first image and original coordinates of the second image match. For example, where the first image is a reference image and the second image is a live angiography feed with a point of origin at the center of the live angiography feed, the distance coordinates would be calculated from the absolute coordinates of the reference image to the center of the reference image. Specifically, in reference angiography image 1120 the absolute coordinates of upper left coordinate of AOI 1131 may be understood as −3, −4 and the distance coordinate of the upper left coordinate of AOI 1131 may be understood as 10, −9. The distance coordinates may be used to convert the remaining coordinates of the AOI. The system may determine the remaining coordinates based on the absolute coordinates of the remaining coordinates, the size and shape of the AOI, etc. For example, if the upper left corner is determined to be 10, −9, the lower right coordinate of the AOI may be determined to be 0, −2.

Once the scale factor has been applied and the distance coordinates have been determined, the system may correlate the distance coordinates of the reference angiography image to the coordinates of the live angiography feed, such that the coordinates are equal. The system may simultaneously manipulate the reference angiography image 1120 and the live angiography feed 1110.

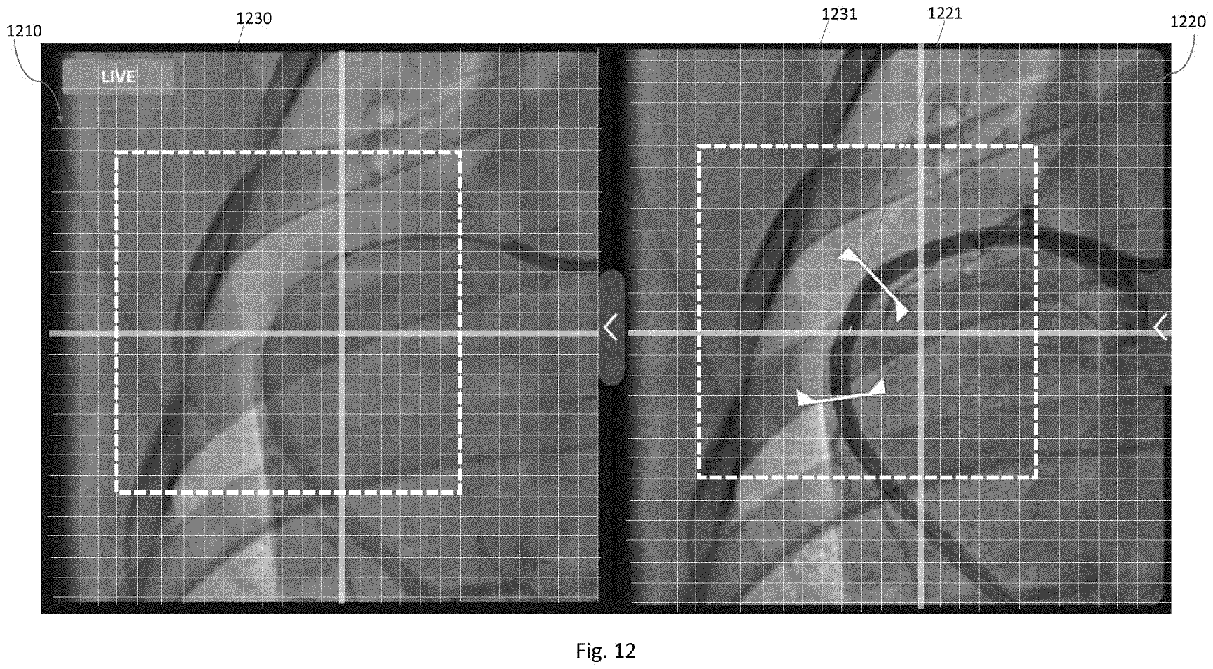

FIG. 12 depicts a sample deployment screen with grid coordinates overlaid onto the screen. The left screen shows the live angiography feed 1210 of the X-ray fluoroscopy. The right screen shows the reference angiography image 1220 with the stent plan marked between the brackets 1221. The depiction shows a deployment screen where both the live angiography feed 1210 and the reference angiography image 1220 have been simultaneously zoomed into an AOI. After converting the coordinates of the reference angiography image 1220, as described above, the system may be able to simultaneously manipulate the live angiography feed 1210 and reference angiography image 1220. The manipulations may be input into the system by a user, such as zoom and pan.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method, comprising:
accessing, using one or more processors, a live imaging feed of a blood vessel;
receiving, using the one or more processors, a reference image of the blood vessel;
aligning, using the one or more processors, the live imaging feed and the reference image such that objects represented in a field of view of the reference image correspond to objects represented in a field of view of the live imaging feed;
receiving user input associated with the reference image;
adjusting, using the one or more processors, the field of view of the reference image based on the user input; and
automatically adjusting, using the one or more processors, the field of view of the live imaging feed based on the user input to the reference image.

2. The method of claim 1, wherein the user input comprises a selected zoom level.

3. The method of claim 2, wherein aligning the live imaging feed and the reference image further comprises:
identifying, using one or more processors, an area of interest on the reference image;
calculating, using one or more processors, a scale factor based on a size of the objects represented in the field of view of the reference image and a size of the objects represented in the field of view of the live imaging feed, wherein the fields of view are associated with a grid;
identifying, using one or more processors, a point of origin of the grid associated with the live imaging feed;
transposing, using one or more processors, the point of origin onto the reference image;
calculating, using one or more processors, a distance coordinate based on the distance between the area of interest and the transposed point of origin; and
converting, using one or more processors, coordinates of the area of interest to match coordinates of the live imaging feed, based on the scale factor and the distance coordinate.

4. The method of claim 3, wherein the scale factor is based on the selected zoom level and is calculated as a ratio of a zoomed reference image and a non-zoomed reference image.

5. The method of claim 3, further comprising simultaneously displaying, using one or more processors, the converted area of interest on the reference image based on the converted coordinates and the live imaging feed.

6. The method of claim 1, wherein object includes at least a portion of a blood vessel.

7. The method of claim 1, wherein the live imaging feed comprises an extravascular image derived from an imaging system based on angiography, fluoroscopy, x-ray, nuclear magnetic resonance, or computer aided tomography.

8. The method of claim 1, wherein the reference image comprises a still extraluminal image co-registered with intravascular data, wherein the intravascular data is derived from an intravascular device that collects data using at least one of optical coherence tomography (OCT), intravascular ultrasound, near-infrared spectroscopy, or micro-OCT.

9. The method of claim 1, wherein the reference image further comprises a surgical guide overlay.

10. The method of claim 1, wherein the adjusting the reference image and the live imaging feed, the user input comprises simultaneously panning the live imaging feed and the reference image.

11. A system, comprising:
one or more processors configured to:
access a live imaging feed of a blood vessel;
receive a reference image of the blood vessel;
align the live imaging feed and the reference image such that objects represented in a field of view of the reference image correspond to objects represented in a field of view of the live imaging feed;
receive user input associated with the reference image;
adjust the field of view of the reference image based on the user input; and
simultaneously adjust the field of view of the live imaging feed based on the user input to the reference image.

12. The system of claim 11, wherein the user input comprises a selected zoom level.

13. The system of claim 12, wherein the one or more processors are further configured to:
identify a selected area of interest on the reference image;
calculate a scale factor based on a size of the objects represented in the field of view of the reference image and a size of the objects represented in the field of view of the live imaging feed, wherein the fields of view are associated with a grid;
identify a point of origin of the grid associated with the live imaging feed;
transposing the point of origin onto the reference image;
calculate a distance coordinate based on the distance between the area of interest and the transposed point of origin; and
convert coordinates of selected area of interest to match coordinates of live imaging feed, based the scale factor and the distance coordinate.

14. The system of claim 13, wherein the scale factor is the scale factor is based on the selected zoom level and is calculated as a ratio of a zoomed reference image and a non-zoomed reference image.

15. The system of claim 13, wherein the one or more processors are further configured to simultaneously display the converted area of interest on the reference image based on the converted coordinates and the live imaging feed.

16. The system of claim 11, wherein object includes at least a portion of a blood vessel.

17. The system of claim 11, wherein the live imaging feed comprises an extravascular image derived from an imaging system based on angiography, fluoroscopy, x-ray, nuclear magnetic resonance, or computer aided tomography.

18. The system of claim 11, wherein the reference image comprises a still extraluminal image co-registered with intravascular data, wherein the intravascular data is derived from an intravascular device, that collects data using at least one of an optical coherence tomography (OCT), intravascular ultrasound, near-infrared spectroscopy, or micro-OCT.

19. The system of claim 11, wherein the reference image further comprises a surgical guide overlay.

20. The system of claim 11, wherein the user input comprises simultaneously panning the live imaging feed and the reference image.

21. A method of providing annotations on a live angiography feed, comprising:
identifying, through an intravascular imaging procedure, one or more portions of a patient's vessel;
matching a frame in the live angiography feed with a frame from the intravascular imaging procedure, such that both frames are captured during corresponding portions of a cardiac cycle of the patient;
correcting rigid displacement, using rigid registration; and
identifying, based on the matched frames and the corrected breathing motion, a spot on an image overlay that corresponds to a portion of the patient's vessel shown in the live angiography feed and the one or more portions identified during the intravascular imaging technique.

22.-42. (canceled)
